(19) United States
(12) Patent Application Publication
(10) Pub. No.: US 2010/0016264 A1
(43) Pub. Date: Jan. 21, 2010

(54) TREATMENT FOR DRY EYE USING TESTOSTERONE AND PROGESTAGEN

(76) Inventors: Charles G. Connor, Germantown, TN (US); Charles Haine, Claremont, CA (US)

Correspondence Address: PATTON BOGGS LLP, 8484 WESTPARK DRIVE, SUITE 900, MCLEAN, VA, 22102, US

(21) Appl. No.: 12/498,166

(22) Filed: Jul. 6, 2009

Related U.S. Application Data
(63) Continuation of application No. PCT/US2007/086517, filed on Dec. 5, 2007.
Application No. PCT/US2007/086517 is a continuation of application No. PCT/US2007/086515, filed on Dec. 5, 2007.

(51) Int. Cl. A61K 31/57

(52) U.S. Cl. 514/170; 514/177

(57) ABSTRACT

The present invention comprises a composition and methods for treating eye conditions using a composition having a therapeutically effective amount of a progestagen, or a therapeutically effective amount of a progestagen with a testosterone; and pharmaceutically acceptable carrier, wherein the composition is applied to the palpebral part of the eye and/or ocular surface.

### TECHNICAL FIELD OF THE INVENTION

The invention generally relates to compositions and methods for treating eye conditions, in particular dry eye with a combination of testosterone and progestagen, wherein the composition is applied to the palpebral part of the eye and/or the ocular surface. The invention also generally relates to compositions and methods for treating eye conditions, in particular dry eye with progestagens, wherein the composition is applied to the palpebral part of the eye and/or the ocular surface.

### BACKGROUND OF THE INVENTION

Dry eye, also known as Keratoconjunctivitis Sicca (“KCS”), is a condition in which the quality and/or quantity of tears bathing the eye decline. People who have dry eye may experience inflammation, dryness and/or foreign body sensation in the conjunctival region of the eye, light sensitivity, itching, burning or stinging, grittiness, tired eyes, contact lens intolerance, and blurring of vision. Almost all dry eye disorders are a result of a loss of water from the tear film. The loss of water from the tear film may be caused by a decrease in tear production and/or an increase in evaporation of tears, which may be a result of an abnormality in mucin or lipid components of the tear film. These phenomena may occur together, but both typically result in increased osmolarity from the normal limit of 311 mOsm/L and may ultimately lead to a decrease in goblet-cell density. A decrease in goblet-cell density affects the production of mucus, which is the major lubricant in the tear film. This aggravates and/or causes inflammation by T-cell activation, resulting in inflammatory cytokines being released.

It has also been shown that patients with chronic dry eye typically experience increased activation of T-cells. These T-cells release cytokines that may result in: (1) neural chemical to the lacrimal gland that disrupt production of natural tears leading to a decrease tear production; (2) tissue damage in the lacrimal glands and/or ocular surface; (3) recruitment of additional T-cells; and/or (4) increased inflammatory cytokine production.

Conditions that may give rise to dry eye include, but are not limited to, Sjögren syndrome, blepharitis, meibomian gland disorder, HIV, herpes zoster, autoimmune disease, the natural aging process, diabetes, long-term contact lens wear, dry environment, surgery that involves corneal incisions or ablates corneal nerves, medications, decreased blinking, eyelids that cannot be closed, pregnancy, polycystic ovary syndrome, acne rosacea, Lupus, Scleroderma, Sarcoidosis, Stevens-Johnson syndrome, Parkinson's, smoking, radiation therapy, vitamin A deficiency, and menopause. This wide divergence in causative factors makes it particularly difficult to fashion a successful treatment for dry eye.

Generally, the tear film is made up of three layers: (1) an innermost hydrophilic mucin layer produced by the conjunctiva goblet cells and the ocular surface epithelium and which serves as an anchor for the tear film, helping it adhere to the eye; (2) a middle thick aqueous layer produced by the lacrimal glands; and (3) a superficial thin lipid layer produced by the meibomian glands, which helps with uniform tear spreading and to slow down tear evaporation. This three-layer structure stabilizes the tear film and enables the tear film to keep the eye moist, create a smooth surface for light to pass through the eye, nourish the front of the eye, and provide protection from injury and infection. The quality of tears in a dry eye sufferer is typically defective with respect to this protective and stabilizing structure.

There are several techniques for diagnosing and evaluating the severity of a patient's dry eye, including the Ocular Surface Disease Index (OSDI) questionnaire, Tear Break-up Time, tear staining, tear film height, and the Schirmer Test. See, Milder, B, The Lacrimal System, Appleton-Century-Crofts, Chapter 8, 1993 and Schirmer, O Studien Zur Physiologie and Pathologie der Tranenabsonderdung und Tranenabfuh, Arch kiln ophthalmol, 1903; 56:197-291, each of which is herein incorporated by reference in its entirety. Each test provides different information about the tear film of a patient.

The patient's subjective evaluation of the severity of the symptoms can be recorded using the standardized OSDI questionnaire. This subjective evaluation can be confirmed by objective indicators such as the Tear Break-up Time (TBUT) test, and the Schirmer Test. The TBUT test measures the time required for the three-layer tear film to separate. A shortened TBUT test time indicates a decreased quality of tears and is indicative of dry eye. See, Lemp et al., Factors Affecting Tear Film Break Up in Normal Eyes, Arch Ophthalmol 1973; 89:103-105, which is herein incorporated by reference in its entirety. The Schirmer Test measures the volume of tears produced, and is performed by of placing a small strip of filter paper inside the lower eyelid (conjunctival sac) of each eye for several minutes, allowing tear fluid to be drawn into the filter paper by capillary action. The paper is then removed and the amount of moisture is measured in millimeters. Typically, a measurement of less than 10 mm in 5 minutes indicates dry eye. See, Schirmer, O Studien zur physiologie and pathologie der tranenabsonderdung und tranenabfuh, Arch kiln ophthalmol, 1903; 56:197-291.

Current treatments for dry eye include artificial tears, and/or ointments and gels for application to the ocular surface. These provide basic lubrication to the eye surface. Restasis® eye drops (cyclosporine in a castor oil base) are said to help the eyes increase tear production. Other treatments include temporary and permanent punctal occlusions, topical androgen eye drops, topical antibiotics, and oral therapy with polyunsaturated fatty acids. For example, U.S. Pat. No. 6,659,985, herein incorporated by reference in its entirety, discloses using androgens for the treatment of dry eye by applying the composition to the adnexa of the eye.

There are drawbacks to the current dry eye treatments. For example, Restasis® is said to have a slow onset of action, appears to help only about 20% of patients, does not appear to work for severe dry eye cases, and has side effects such as burning on instillation. With punctal plugs, infection may occur and surgical removal may be required. Topical administration of steroids may have adverse effects such as increase in intraocular pressure, glaucoma, cataract, and exacerbation of corneal infection. See Butcher, et al., Bilateral Cataracts and Glaucoma Induced by Long Term Use of Steroid Eye Drops, BMJ, 1994; 309:43, which is herein incorporated by reference in its entirety.

There are three types of sex steroid hormones: androgens, estrogens, and progestagens. Androgens are natural or synthetic steroid hormones that stimulate or control the development and maintenance of masculine characteristics in vertebrates by binding to androgen receptors. This includes the activity of the accessory male sex organs and development of male secondary sex characteristics, such as testis formation and spermatogensis. Androgens are also the original anabolic steroids, and assist in inhibition of fat deposition and increased muscle mass. They are also the precursor of all estrogens, the female sex hormones. The primary and most well-known androgen is testosterone, which has been used to treat dry eye.

The progestagens are hormones which have progestational activity, i.e., produce effects similar to progesterone (the only natural progestagen), such as preparing the uterus for the reception and development of the fertilized ovum by transforming the endometrium from the proliferative to the secretory stage and maintaining an optimal intrauterine environment for sustaining pregnancy. Progesterone is both a final product of the steroid hormone pathway as well as an intermediate in the synthesis of cortisol. This pathway occurs in both men and women. In women, progesterone is produced in the corpus luteum of the ovary as well as the placenta. It is also produced in the adrenal cortex in both sexes. Progesterone, in contrast to estrogen, is mildly catabolic in humans and can be thought of as balancing the action of estrogen. The biological actions of progesterone are diverse and often opposing. Its effect on target tissues is mediated by progesterone receptors that function as ligand-activated transcription factors to regulate the expression of specific sets of target genes. The progesterone receptor belongs to a large family of nuclear receptors which include receptors for the following: (i) steroid hormones (estrogen, progesterone, glucocorticoid, androgen, and mineralcorticoid); (ii) other lipophilic hormones and ligands (thyroid hormone, retinoic acid, 9-cis retinoic acid, vitamin D³, eicosanoids, fatty acids, and lipids); and (iii) orphan receptors that have no known ligand. The progesterone receptor and corticosteroid receptor share regions of high homology, particularly within the DNA-binding domain of the steroid hormone receptor family which results in cross reactivity. The precise physiological effects of progestagens can be difficult to interpret due to their potential to cross-react with other nuclear receptors, such as glucocorticoid, mineralcorticoid, and androgen receptors.

Progestagens may have cross-reactivity with other sex hormones such as by acting on different types of receptors, but with respect to the present invention, progestagens are those molecules that predominantly have progestational activity.

Progestagens are currently used: (1) in the prevention of miscarriage; (2) to treat various cancers, such as breast, kidney, and uterine; (3) to treat menstrual disorders and other gynecological disorders; (4) as an oral contraceptive; (5) in systemic hormone replacement therapy (HRT); (6) to treat loss of appetite and severe weight and/or muscle loss due to AIDS and/or cancer; and (7) as an antiandrogen. In the treatment of these disorders progestagens are used in many forms such as pills, injections, vaginal suppositories, and skin creams.

The inventors are credited with discovering that use of progestagens, or a combination of progestagens with testosterone, unexpectedly results in improving treatment of dry eye. Up until the present invention, progestagens have not been used to treat dry eye. Further, no treatment currently exists where a composition having at least one progestagen, or a combination of progestagen and testosterone, is applied to the palpebral part of an eye and/or the ocular surface to treat dry eye.

We have discovered that transdermal treatments of the eye with a therapeutically effective amount of progestagen, or progestagen and testosterone, are surprisingly effective at alleviating certain eye diseases, particularly dry eye. While not wishing to be constrained to any presently understood mode of action, it is believed this effect is generally independent of systemic hormone activity. Thus, surprisingly effective results can be obtained with low levels of hormone. The aim of this immunoendocrine interaction is to: (a) reduce lymphocyte infiltration in adjacent lacrimal tissue and thereby alleviate immune-mediated destruction, and lymphocyte compression, of acinar and ductal cells; (b) permit accessory and/or palpebral lacrimal glands to secrete basal tear volumes; and (c) avoid the side effects that parallel systemic exposure to these hormones. In effect, transdermal treatment of the composition can generate functional regions of lacrimal tissue, thereby enhancing tear output and correcting certain eye conditions, particularly dry eye.

### SUMMARY OF THE INVENTION

The present invention relates to compositions delivery options and methods for treating eye conditions, and in particular dry eye, wherein the composition contains a therapeutically effective amount of a progestagen, or a combination of progestagen and testosterone, with at least one pharmaceutically acceptable carrier. Such conditions can also include the effects resulting from laser or other types of eye surgery.

It is an object of the invention to formulate the composition such that it minimizes or avoids systemic treatment of the individual with progestagen, or a combination of progestagen and testosterone. Further, the novel administration of the progestagen, or a combination of progestagen and testosterone, avoids the disadvantages encountered with oral drug administration, e.g., degradation of the drug by fluids present in the gastrointestinal tract and/or first-pass inactivation in the liver.

Further, the invention relates to compositions and methods for transdermal treatment of dry eye wherein the composition has a therapeutically effective amount of progestagen. Other embodiments related to composition with a therapeutically effective amount of progestagen and testosterone. The amount of progestagen and testosterone will vary based upon the desired treatment amount, severity of the eye disease, and carrier used in the formulation of the composition. Further, the pharmaceutically acceptable carrier may include any carrier known in the art for use with topical application to the skin and transdermal delivery of a sex steroid hormone, or which is known to be suitable for delivery to the conjunctiva. Application of the progestagen, or a combination of progestagen and testosterone, to the palpebral part of the eye will can act directly on accessory and main lacrimal tissues and suppress the glandular inflammation in these tissues.

It is an object of certain embodiments of the invention to prepare the composition for treating dry eye where in the composition has a therapeutically effective amount of progestagen, or a combination of progestagen and testosterone.

In one embodiment, the composition contains a therapeutically effective amount of a progestagen with at least one pharmaceutically acceptable carrier and is developed for transdermal application. In another embodiment, the composition contains a therapeutically effective amount of a progestagen and testosterone with at least one pharmaceutically acceptable carrier and is developed for transdermal application. The compositions are a transdermal formulation that is to be applied to the palpebral part of the eye, which includes the upper and lower eyelids and the medial and literal canthus. Transdermal application to the palpebral part of the eye is preferred, as the progestagen and testosterone appears to be easily absorbed across the skin where it may then interact with the target gland.

In another embodiment, the composition contains a therapeutically effective amount of a progestagen and at least one pharmaceutically acceptable carrier and is formulated to be applied to the ocular surface, which includes the conjuntiva.

In another embodiment, the composition contains a therapeutically effective amount of a progestagen and testosterone and at least one pharmaceutically acceptable carrier and is formulated to be applied to the ocular surface, which includes the conjuntiva.

In other embodiments, the composition contains a therapeutically effective amount of a progestagen, at least one pharmaceutically acceptable carrier, and at least one estrogen, and is developed for transdermal application.

In other embodiments, the composition contains a therapeutically effective amount of a progestagen and testosterone, at least one pharmaceutically acceptable carrier, and at least one estrogen, and is developed for transdermal application.

In one embodiment, the composition contains a therapeutically effective amount of a progestagen, at least one pharmaceutically acceptable carrier, and at least one estrogen, and is developed for ocular surface application.

In one embodiment, the composition contains a therapeutically effective amount of a progestagen and testosterone, at least one pharmaceutically acceptable carrier, and at least one estrogen, and is developed for ocular surface application.

### DETAILED DESCRIPTION

Reference will now be made in detail to embodiments of the invention. While the invention will be described in conjunction with the embodiments, it will be understood that they are not intended to limit the invention to those embodiments. On the contrary, the invention is intended to cover alternatives, modifications, and equivalents, which may be included within the spirit and scope of the invention as defined by the appended claims.

As referred to herein, the term “progestagen” includes but is not limited to natural and synthetic progesterone, natural and synthetic progestagens (which are sometimes referred to in the art as “progestins”), medroxyprogesterone acetate (medrysone), norethindrone (or norethisterone), norethindrone acetate, megestrol acetate, 17-a-hydroxyprogesterone caproate, and norgestrel, and derivatives thereof. Natural progesterone does not have any serious clinical side effects nor have any toxic levels been identified. Further, progestagen includes the three forms of progesterone recognized by the U.S. Pharmacopoeia, namely progesterone USP micronized, progesterone USP wettable microcrystalline, and progesterone USP milled. Each of these forms may be used with the present invention, preferably progesterone USP milled.

As used herein, the term “testosterone” refers to the compound having the IUPAC names (17)-17-Hydroxyandrost-4-en-3-one, and delta-4-androsten-17-ol-3-one, as well as their isomers. Testosterone is listed in the Merck Index, entry no. 9322, at page 1569, 12th ed., (1996), which is incorporated herein by reference. Testosterone may be obtained or prepared using the knowledge of one ordinarily skilled in the art from either a natural source, or synthetically using a process. Further, the term “testosterone” also refers to a number of closely related androgenic compounds which are synthetically derivatized from testosterone are known to provide the same or a similar physiologic activity. Such compounds include, but are not limited to, testosterone salts, such as acetate, enanthate, cypionate, isobutyrate, propionate, and undecanoate esters, cyproterone acetate, danazol, finasteride, fluoxymesterone, methyltestosterone, nandrolone decanoate, nandrolone phenpropionate, oxandrolone, oxymetholone, stanozolol, and testolactone.

As used herein, the terms “estrogen”, and “estrogenic hormone” refer to any substance, natural or synthetic, that exerts a biological or pharmacological action primarily by binding to estrogen receptors. Examples include, but are not limited to, 17-β-estradiol, 17-α-estradiol, estriol, estrone, and phytoestrogens. These estrogens may be derivatized or modified to form, for example, conjugated equine estrogens, esterified estrogens, ethinyl estradiol, etc. Examples of esterified estrogens include but are not limited to: estradiol-3,17-diacetate, estradiol-3-acetate, estradiol-17-acetate, estradiol-3,17-divalerate, estradiol-3-valerate, estradiol-17-valerate. Also included are selective estrogen receptor modulators (SERMS), for example raloxifene, available under the tradename Evista®. from Eli Lilly, and the like. The estrogens may also be present as salts, e.g., as sodium estrogen sulfate, isomers, or prodrugs.

As used herein, the term “palpebral part of an eye” is the external portion of the upper and lower eyelids and the medial and lateral canthus.

As used herein, “administration,” and “administering” may be used interchangeably, and refer to the act of presenting, applying, or introducing a drug to a subject to achieve a desired physiological response.

As used herein, “carrier,” and “pharmaceutically acceptable carrier” may be used interchangeably, and refer to any liquid, gel, salve, solvent, liquid, diluent, fluid ointment base, liposome, micelle, giant micelle, and the like, which is suitable for use in contact with living animal or human tissue without causing adverse physiological responses, and which does not interact with the other components of the composition in a deleterious manner. A number of carrier ingredients are known for use in making topical formulations, such as gelatin, polymers, fats and oils, lecithin, collagens, alcohols, water, etc.

As used herein, “disease” and “condition” may be used interchangeably, and refer to one or more physical or psychological signs, symptoms, or laboratory findings, which indicate an illness, deficiency, or other abnormal state of well being.

The terms “formulation” and “composition” are used interchangeably herein and may be in any form usable to the practitioner, including a gel, a cream, a lotion, a solution or an ointment.

As used herein, “skin,” “skin surface,” “derma,” “epidermis,” and similar terms are used interchangeably herein, and refer to only the outer skin of the palpebral part of an eye of a subject comprising the epidermis.

As used herein, “effective amount” or “pharmacologically effective amount” refers to an amount of a substance which is sufficient to achieve its intended purpose or effect. Various biological factors may affect the ability of a delivered substance to perform its intended task. Therefore, an “effective amount” may be dependent on such biological factors. Included among those factors are the carrier used, the tolerance for the active ingredient, the response elicited, the number of unit dose administrations desired to be used the age, size, and gender of the recipient, as well as other medicaments used by the recipient. Further, determination of the effectiveness of the amount is well within the knowledge and ability of one of ordinary skill in the art.

As used herein, “percent by weight” and “% w/w” refer to the amount of an indicated component with respect to an entire composition of which the component is a part. By way of example, progesterone in an amount of 20% w/w refers to the amount of progesterone being 20% of the weight of the total formulation which contains the progesterone.

The terms “topical formulation” and “transdermal formulation” means a composition in which the progestagen, or combination of progestagen and testosterone, may be placed for direct application to a skin surface and from which an effective amount of progestagen, or combination of progestagen and testosterone, is released to the skin surface. Examples of topical formulations include but are not limited to ointments, creams, gels, transdermal patches, sprays, and pastes.

The term “transdermal” refers to the route of administration that facilitates transfer of a progestagen, or a combination of progestagen and testosterone, through a skin surface wherein a transdermal composition is administered to the skin surface. Transdermal administration can be accomplished by applying, pasting, rolling, attaching, pouring, pressing, rubbing, etc., of a transdermal preparation onto a skin surface.

As used herein, “therapeutic effect” refers to a desired result which is achieved to some degree.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

### PREFERRED EMBODIMENTS

The present invention is to methods of making and using compositions having a therapeutically effective amount of a progestagen, or combination of progestagen and testosterone, with a pharmaceutically acceptable carrier. According to a preferred embodiment, the composition is used to treat dry eye. One embodiment is to a composition and its use in treating dry eye wherein the composition comprises a therapeutically effective amount of a progestagen and testosterone with a pharmaceutically acceptable carrier. One embodiment is to a composition and its use in treating dry eye wherein the composition comprises a therapeutically effective amount of a progestagen with a pharmaceutically acceptable carrier. In one preferred embodiment, the composition is prepared for transdermal use. In another preferred embodiment, the composition is prepared for topical application onto the ocular surface of the eye.

### Hormones

The amount of progestagen, or combination of progestagen and testosterone, that is to be administered depends on the age of the patient, duration of the disease state, the particular condition to be treated, the frequency of administration, and the route of administration.

Further, the amount of progestagen, or combination of progestagen and testosterone, in the composition will vary depending upon the pharmaceutically acceptable carrier used and the desired concentration delivered to a patient for treatment.

Transdermal delivery and topical application of the progestagen, or combination of progestagen and testosterone, composition will have little or no systemic side effects typically caused by oral use and/or injection of steroid hormones. The concentration of the progestagen, or combination of progestagen and testosterone, composition should be high enough to affect the area to which the composition is applied as well as the targeted structures of the eye, but low enough to prevent the typical side effects associated with systemic hormone treatments.

When applied to the palpebral region, the amount of progestagen in the composition may range from about 2% to about 30%. In another embodiment, the amount of progestagen in the composition may range from about 10% to about 20%. In yet another embodiment, the amount of progestagen in the composition may range from about 12% to about 18%. In another embodiment, the amount of progestagen in the composition may range from about 10% to about 30%. In yet another embodiment, the amount of progestagen in the composition may range from about 15% to about 25%. In another embodiment, the amount of progestagen in the composition is about 15%.

When applied to the ocular surface, the amount of progestagen in the composition may range from about 0.001% to 20% by weight. In one embodiment, the amount of progestagen in a composition for application to the ocular surface may range from about 0.1% to about 10% by weight. In another embodiment, the amount of progestagen in the composition for application to the ocular surface may range from about 0.4% to about 6% by weight. In another embodiment, the amount of progestagen in the composition for application to the ocular surface may range from about 0.8% to about 5% by weight. In yet another embodiment, the amount of progestagen in a composition for application to the ocular surface is about 2%. In yet another embodiment, the amount of progestagen in a composition for application to the ocular surface is about 5%

When applied to the palpebral region, the amount of testosterone in the composition may range from about 0.01% to about 30%. In another embodiment, the amount of testosterone in the composition may range from about 1% to about 5%. In yet another embodiment, the amount of testosterone in the composition is about 15%.

When applied to the ocular surface, the amount of testosterone in the composition may range from about 0.001% to 20% by weight. In one embodiment, the amount of testosterone in a composition for application to the ocular surface may range from about 0.1% to about 10% by weight preferably about 0.4% to about 6%, more preferably about 0.8% to about 5%. In yet another embodiment, the amount of testosterone in a composition for application to the ocular surface is about 2%.

The composition may be applied once or more a day, depending upon, but not limited to, the needs of the patient and/or the severity of the condition. In one embodiment, the composition is applied once a day. In another embodiment, the composition is applied twice a day. The amount of the progestagen, or amount of progestagen and testosterone when combined, in the composition that is applied to each eye per day will vary depending on, but not limited to, the severity of the dry eye and/or number of applications. In one embodiment, the amount of the progestagen composition applied to each eye per day ranges from about 25 mg to about 500 mg. In an alternate embodiment, the amount of the progestagen composition that is applied to each eye per day is from about 100 mg to about 400 mg. In yet an alternate embodiment, the amount of the progestagen composition that is applied to each eye per day is from more preferably about 160 mg. In one embodiment, the amount of the progestagen and testosterone composition applied to each eye per day ranges from about 25 mg to about 500 mg. In an alternate embodiment, the amount of the progestagen and testosterone composition that is applied to each eye per day is from about 100 mg to about 400 mg. In yet an alternate embodiment, the amount of the progestagen and testosterone composition that is applied to each eye per day is from more preferably about 160 mg.

The present invention may also be used in combination with other skin treatment ingredients, such as but not limited to sunscreen, vitamins, plant extracts, and moisturizers.

The progestagen, or combination of progestagen and testosterone, may be prepared for inclusion in the composition of the present invention by use of liposomes or microemulsions. The progestagen, or combination of progestagen and testosterone, may be encapsulated in liposomes, thereby creating a delivery vehicle with a consistent absorption rate. Microemulsions may also be used as a delivery vehicle for progesterone and testosterone. See Paul, et al., Curr. Sci., Apr. 25, 2001, 80(8):990-1001, which is herein incorporated by reference in its entirety.

In one embodiment, the composition further comprises at least one estrogen. The amount of estrogen that is to be administered can depend on the age of the patient, duration of the disease state, the particular condition to be treated, the frequency of administration, and the route of administration. Further, the amount of estrogen in the composition will vary depending upon the pharmaceutically acceptable carrier used and the desired concentration delivered to a patient for treatment. In one embodiment, the amount of estrogen is very low or di minimus. In one embodiment, the amount of estrogen in the composition may range from about 0.01% to about 30%. In one embodiment, the amount of estrogen in the composition may range from about 0.25% to about 10%. In one embodiment, the amount of estrogen in the composition may range from about 0.25% to about 5%. In one embodiment, the amount of estrogen in the composition is about 0.25%.

### Pharmaceutically Acceptable Carriers

Pharmaceutically acceptable carriers for use with the formulations of the present invention are well known in the cosmetic and pharmaceutical arts, and include—but are not limited to—such vehicles as water; organic solvents, alcohols, lower alcohols that are readily capable of evaporating from the skin, ethanol, glycols, glycerin, aliphatic alcohols, mixtures of water and organic solvents, mixtures of water and alcohol, mixtures of organic solvents such as alcohol and glycerin, lipid-based materials such as fatty acids, acylglycerols, oils, mineral oils, fats of natural or synthetic origin, phosphoglycerides, sphingolipids, waxes, DMSO, protein-based materials such as collagen and gelatin, volatile and/or non-volatile silicon-based materials, cyclomethicone, demethiconol, dimethicone copolyol (Dow Corning), hydrocarbon-based materials such as petrolatum and squalane, sustained-release vehicles such as microsponges and polymer matrices, suspending agents, emulsifying agents, and other vehicles and vehicle components that are suitable for administration to the skin, as well as mixtures of topical vehicle components as identified above or otherwise known to the art.

The pharmaceutically acceptable carrier may also be a commercially available neutral base known in the art. A neutral base has no significant therapeutic effect of its own. It simply conveys the active pharmaceutical ingredient, although some vehicles may do so with greater ease or effectiveness than others. A neutral base may be a cream used cosmetically for softening and/or cleaning the skin. Examples include Eucerin® (Beiersdorf Aktiengesellschaft Corp., Hamburg, Germany), Aquaphor® (Beiersdorf Aktiengesellschaft Corp., Hamburg, Germany), and liposomal vehicles. A preferred neutral base is Vanicream® (Pharmaceutical Specialties, Inc., Rochester, Minn.). Vanicream® is composed of purified water, white petrolatum, cetearyl alcohol and ceteareth-20, sorbitol solution, propylene glycol, simethicone, glyceryl monostearate, polyethylene glycol monostearate, sorbic acid and butylated hydroxytoluene (BHT).

The pharmaceutically acceptable carrier may be a transdermal gel such as Pluronic Lecithin Organogel (PLO). See, Murdan, A Review of Pluronic Lecithin Organogel as a Topical and Transdermal Drug Delivery System, Hospital Pharmacist, July/August 2005, Vol. 12, pp. 267-270, which is herein incorporated by reference in its entirety.

In some embodiments, the pharmaceutical acceptable carrier also includes at least one surfactant. The surfactant may be selected from, but is not limited to, anionic, cationic, amphoteric, zwitterionic, and nonionic surfactants. If the surfactant is nonionic, it may be selected from the group consisting of: polysorbates, poloxamers, alcohol ethoxylates, ethylene glycol-propylene glycol block copolymers, fatty acid amides, alkylphenol ethoxylates, or phospholipids.

In some embodiments, the pharmaceutical acceptable carrier also includes a chelating agent, including but not limited to, edetate salts, like edetate disodium, edetate calcium disodium, edetate sodium, edetate trisodium, and edetate dipotassium.

In some embodiments, the pharmaceutical acceptable carrier also includes viscosity enhancing agents to delay wash-out or wash-off, such methyl cellulose, hydroxyethyl cellulose, hydroxypropylmethyl cellulose, carboxymethyl cellulose, polyethyleneoxide, and dextrans.

In one embodiment, one or more penetration enhancers may be included in the composition of the present invention. The types of penetration enhancers include, but are not limited to, phospholipids, terpenes, anionic surfactants, cationic surfactants, zwitterionic surfactants, nonionic surfactants, fatty acids, fatty esters, fatty amines, azone-like compounds, sodium salts of fatty acids, polyethylene glycol monolaurate (“PEGML”), glycerol monolaurate, lecithin, the 1-substituted azacycloheptan-2-ones, particularly 1-n-dodecylcyclaza-cycloheptan-2-one (available under the trademark Azone® from Nelson Research & Development Co., Irvine, Calif.), lower alkanols (e.g., ethanol), SEPA®, cholic acid, taurocholic acid, bile salt type enhancers, and surfactants such as Tergitol®, Nonoxynol-9® and TWEEN-80®,

Some embodiments further include one or more preservatives to be used when the composition. The preservative may be desired for many reasons, including increasing shelf life, protecting the composition from chemical change, and protecting the composition from microbial action. The term preservative has the meaning commonly understood in the ophthalmic art. Preservatives may be used to prevent bacterial contamination in multiple-use ophthalmic preparations, and, while not intending to be limiting, examples include benzalkonium chloride, stabilized oxychloro complexes (otherwise known as Purite®), phenylmercuric acetate, chlorobutanol, benzyl alcohol, parabens, anti-oxidants, anti-microbials, anti-fungals, and thimerosal.

The pharmaceutically acceptable carrier may further include components adapted to improve the stability or effectiveness of the applied formulation, such as preservatives, antioxidants, skin penetration enhancers, sustained release materials, and the like. Examples of such vehicles and vehicle components are well known in the art.

### Method of Use

The topical application of a progestagen, or combination of progestagen and testosterone, composition of the present invention to the palpebral part of the eye allows for easy application and for transdermal delivery of the active ingredient to the sites of action. The sites of action may include, but are not limited to, the ocular surface, which includes the cornea and conjunctiva; the lacrimal gland and lacrimal accessory glands; and the meibomian glands. This form of transdermal delivery provides effective treatment without the side effects caused by systemic use of the drug. These side effects of systemic use of progestagen, or combination of progestagen and testosterone, may include, but are not limited to, upset stomach, cramps, breast tenderness, drowsiness, dizziness, headache, migraine headache, vomiting, diarrhea, constipation, tiredness, skin rash, and lower levels of high density lipoprotein (HDL).

The topical composition of the present invention may also be applied to the ocular surface (as distinguished from the palpebral region), which includes the cornea and conjunctiva. In this case, the composition is typically in the form of drops or an ointment. The topical application of the present invention to the ocular surface may be applied once a day or more frequently based upon, but not limited to, the needs of the patient and/or the severity of the condition. In one embodiment, the topical application is applied to the ocular surface about two to about three times a day. In another embodiment, the topical application is applied to the ocular surface between about 4 times to about 8 times per day. A few drops of the progestagen, or combination of progestagen and testosterone, composition may be applied to the ocular surface as needed for each application.

Without being limited to any particular theory, it is believed that the topical application of the progestagen, or combination of progestagen and testosterone, composition to the palpebral part of the eye permits transdermal delivery of the active ingredient to the areas affected by dry eye diseases, including but not limited to, the lacrimal gland and accessory lacrimal glands. Further, the progestagen may act upon the progesterone receptors located in the lacrimal gland and lacrimal accessory glands, as well as other areas of the eye. Additionally, the progestagen may reduce viable T-cells due to apoptosis which in turn decreases the inflammatory state of the ocular surface and/or eyelids.

Typically, patients experience an improvement of their dry eye symptoms within about 3-7 days of initiation of treatment, and achieve a steady state within about 7 days. The profile of improvement, however, will depend upon the amount of hormone used and its frequency in application.

In some embodiments, the composition contains one or more second therapeutically active agents. The second therapeutically active agent could be any drug which might be useful in treating the symptoms of dry eye, or any of its underlying causes. In addition, the second therapeutically active agent could be any drug which is useful in preventing or treating any disease which might occur simultaneously to dry eye disease, whether or not the disease is related. In another useful aspect of this invention, the second therapeutically active agent could be a drug which is used in topical ophthalmic compositions which might cause, contribute to, or aggravate dry eye disease as a side effect of its use. In this aspect, this invention is useful in reducing or eliminating said side effect.

The one or more second therapeutically active agent may be selected from, but is not limited to, nucleotide purinergic receptor agonists such as uridine 5′-triphosphate, dinucleotides, cytidine 5′-diphosphosphate, adenosine 5′-diphosphate, P1-(cytidine 5′-)-P-(uridine 5′-)tetraphosphates, P1, P4-di(uridine 5′)-tetraphosphates, or their therapeutically effective analogues or derivatives, which may affect tear secretion, particularly the mucous layer of tears, and thus may have potential in treating dry eye disease.

The one or more second therapeutically active agent may be selected from, but is not limited to, nicotinic receptor agonists such as nicotine and its analogs, trans-metanicotine and its analogs, epibatidine and its analogs, pyridol derivatives, piperidine alkaloids such as lobeline and its analogs, certain para-alkylthiophenol derivatives, and imidacloprid and its analogs, which are believed to stimulate secretion of mucin by the conjunctival goblet cells, and thus may be useful in treating dry eye.

The one or more second therapeutically active agents may be selected from, but is not limited to, tetracycline, derivatives or analogues of tetracycline, or chemically modified tetracycline, which are believed to assist in correcting delayed tear clearance.

The one or more second therapeutically active agents may be selected from, but is not limited to, corticosteroids such as methylprednisolone sodium succinate, prednisolone acetate, prednisolone sodium phosphate, fluorometholone, fluorometholone acetate, dexamethasone sodium phosphate, hydroxymethylprogesterone, rimexolane, budesonide, and tixocortol pivalatein, which are believed to be useful in treating dry eye.

The one or more second therapeutically active agents may be selected from, but is not limited to, products of human lacrimal gland acinar epithelia such as growth factors or cytokines including the transforming growth factor beta (TGF-beta), which may to be useful in treating dry eye.

The one or more second therapeutically active agents may be selected from, but is not limited to, cyclosporin and cyclosporin derivatives, such as cyclosporin A, cyclosporin B, cyclosporin C, cyclosporin D, and cyclosporin G.

Although the present invention has been described in terms of specific embodiments, changes and modifications can be made out without departing from the scope of the invention which is intended to be defined only by the scope of the claims.

### EXAMPLE 1

Thirty (30) patients with dry eye symptoms are tested using the Tear Break-up Time Test and Schirmer test with anesthetic to determine the effectiveness of a progesterone composition. The patients also complete the OSDI questionnaire to assess the patient's perception of dry eye severity. The intraocular pressure for each patient also is also determined before and after application of the progesterone composition. The progesterone composition is 15% progesterone in Vanicream®.

Each patient is instructed to cleanse their eyelids prior to applying the progesterone composition. A small amount of the cream, about 50 mg to about 100 mg, is applied to the upper and lower eyelids for each eye until the cream was no longer visible. The cream is applied twice a day, once in the morning and once at bedtime.

The average baseline testing scores is as follows:

| Test Performed | Average Score |
|---|---|
| Tear Break-up Test* | 5.69 |
| Schirmer Test* | 11.90 |
| Intraocular Pressure* | 14.20 |
| OSDI | 28.0 |

The scores for the left and right eyes is averaged to obtain one value for each patient.

The testing scores after three weeks of treatment is as follows:

| Test Performed | Average Score |
|---|---|
| Tear Break-up Test* | 8.0 |
| Schirmer Test* | 14.20 |
| Intraocular Pressure* | 13.86 |
| OSDI | 22.0 |

The scores for the left and right eyes is averaged to obtain one value for each patient.

The TBUT test shows a significant improvement after three weeks of treatment with a p-value of 0.01. The Schirmer test shows a positive trend towards improvement, but did not reach significant significance. The Intraocular Pressure Test shows no change in intraocular pressure. Patients report (OSDI) a perceived improvement in their dry eye symptoms after use of the progesterone cream, with a p-value of 0.05 associated with a 21% improvement in symptoms after three weeks of treatment.

None of the patients report any side effects from use of the progesterone cream and no allergic reactions are reported.

What is claimed is:

1. A composition for treating dry eye, comprising:
a therapeutically effective amount of a progestagen; and
a pharmaceutically acceptable carrier;
wherein the composition is applied to a palpebral part of an eye.

2. The composition of claim 1, wherein the progestagen is progesterone.

3. The composition of claim 1, wherein the composition is applied in an amount between about 25 mg and about 500 mg.

4 The composition of claim 1, wherein the progestagen is present in a concentration from about 2% to about 30%.

5. The composition of claim 1, wherein the composition is applied once or twice a day.

6. The composition of claim 1, wherein about 50 mg to about 100 mg of the composition is applied to each eye.

7. A composition for treating dry eye, comprising:
a progestagen, wherein a treatment amount of the progestagen is present in a concentration of about 15% and
a pharmaceutically acceptable cream carrier;
wherein the composition is applied to a palpebral part of an eye.

8. A composition for treating dry eye, comprising:
a progestagen, wherein a treatment amount of the progestagen is present in a concentration between about 2% and about 30%; and
a pharmaceutically acceptable cream carrier;
wherein the composition is applied to a palpebral part of an eye.

9. A composition for treating dry eye, comprising:
a therapeutically effective amount of a progestagen; and
a pharmaceutically acceptable carrier;
wherein the composition is applied to an ocular surface of an eye.

10. A composition for treating dry eye comprising:
a progestagen, wherein the treatment amount of the progestagen is present in a concentration between about 0.01% and about 10%; and
a pharmaceutically acceptable carrier;
wherein the composition is applied to an ocular surface of an eye.

11. A composition for treating dry eye, comprising:
a therapeutically effective amount of a progestagen;
a therapeutically effective amount of a testosterone; and
a pharmaceutically acceptable carrier;
wherein the composition is applied to a palpebral part of an eye.

12. The composition of claim 11, wherein the progestagen is progesterone.

13. The composition of claim 11, wherein the composition is applied in an amount between about 25 mg and about 500 mg.

14 The composition of claim 11, wherein the progestagen is present in a concentration from about 2% to about 30%.

15. The composition of claim 11, wherein the composition is applied once or twice a day.

16. The composition of claim 11, wherein about 50 mg to about 100 mg of the composition is applied to each eye.

17. The composition of claim 11, wherein said testosterone is selected from the group consisting of: (17)-17-Hydroxyandrost-4-en-3-one, (17)-17-Hydroxyandrost-4-en-3-one isolmers, delta-4-androsten-17-ol-3-one, delta-4-androsten-17-ol-3-one isomers, and mixtures thereof.

18. A composition for treating dry eye, comprising:
a progestagen, wherein a treatment amount of the progestagen is present in a concentration between about 2% and about 30%;
a testosterone, wherein a treatment amount of the testosterone is present in a concentration between about 0.01% and 30%; and
a pharmaceutically acceptable cream carrier;
wherein the composition is applied to a palpebral part of an eye.

19. A composition for treating dry eye, comprising:
a therapeutically effective amount of a progestagen;
a therapeutically effective amount of a testosterone; and
a pharmaceutically acceptable carrier;
wherein the composition is applied to an ocular surface of an eye.

20. A composition for treating dry eye comprising:
a progestagen, wherein the treatment amount of the progestagen is present in a concentration between about 0.01% and about 10%;
a testosterone, wherein the treatment amount of the testosterone is present in a concentration between about 0.001% and 20% and
a pharmaceutically acceptable carrier;
wherein the composition is applied to an ocular surface of an eye.
